(19) United States
(12) Patent Application Publication
(10) Pub. No.: US 2020/0038087 A1
(43) Pub. Date: Feb. 6, 2020

(54) PRESSURE CONTROL ASSEMBLY FOR CRYOGENIC BALLOON CATHETER SYSTEM

(71) Applicant: Boston Scientific Scimed Inc., Maple Grove, MN (US)

(72) Inventor: Chadi Harmouche, Saint Laurent (CA)

(21) Appl. No.: 16/599,448

(22) Filed: Oct. 11, 2019

Related U.S. Application Data
(63) Continuation of application No. PCT/US2018/024750, filed on Mar. 28, 2018.
(60) Provisional application No. 62/484,321, filed on Apr. 11, 2017.

(51) Int. Cl. A61B 18/02

(52) U.S. Cl. CPC A61B 18/02; A61B 2018/0022; A61B 2018/0212

(57) ABSTRACT

A cryogenic balloon catheter system for treating a condition in a patient includes a balloon catheter and an inter-balloon pressure control assembly. The balloon catheter includes a first balloon; and a second balloon that substantially encircles the first balloon to define an inter-balloon space between the first balloon and the second balloon, the inter-balloon space having an inter-balloon pressure. The inter-balloon pressure control assembly controls the inter-balloon pressure in the inter-balloon space. The inter-balloon pressure control assembly includes a vacuum pump that is configured to selectively evacuate fluid from the inter-balloon space to adjust the inter-balloon pressure; and a solenoid valve that is in fluid communication with the inter-balloon space, the solenoid valve selectively allowing the vacuum pump to evacuate the fluid from the inter-balloon space.

### CROSS-REFERENCE TO RELATED APPLICATION

This application is a continuation of International Application No. PCT/US18/24750, with an international filing date of Mar. 28, 2018, which claims priority of U.S. Provisional Application Ser. No. 62/484,321, filed on Apr. 11, 2017, and entitled “ACTIVELY CONTROLLED VALVE FOR CRYOGENIC BALLOON CATHETER ASSEMBLY”. As far as permitted, the contents of International Application No. PCT/US18/24750 and U.S. Provisional Application Ser. No. 62/484,321 are incorporated in their entirety herein by reference.

### TECHNICAL FIELD

The present disclosure relates to medical devices and methods for performing cryoablation procedures. More specifically, the disclosure relates to devices and methods for controlling inter-balloon pressure in a dual-balloon cryoablation catheter.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the user or operator) portion of the catheter, and often at the tip of the catheter. Various forms of energy can be used to ablate diseased heart tissue. These can include radio frequency (RF), cryogenics, ultrasound and laser energy, to name a few. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of the energy delivered is a critical factor in increasing the likelihood that the treated tissue is permanently incapable of conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis while avoiding excessive energy leading to collateral tissue injury.

Atrial fibrillation (AF) is one of the most common arrhythmias treated using catheter ablation. In the earliest stages of the disease, paroxysmal AF, the treatment strategy involves isolating the pulmonary veins from the left atrial chamber. Recently, the use of techniques known as “balloon cryotherapy” catheter procedures to treat AF has increased. In part, this stems from the balloon cryotherapy's ease of use, shorter procedure times and improved patient outcomes. Despite these advantages, there remains needed improvement to further improve patient outcomes and to better facilitate real-time physiological monitoring of tissue to optimally titrate energy to perform both reversible “ice mapping” and permanent tissue ablation.

In balloon cryotherapy systems, it is common that two balloons are used to create a cryo-chamber near the distal tip of the catheter. The balloons are configured such that there is an inner balloon that receives the cryogenic cooling fluid and an outer balloon that surrounds the inner balloon. The outer balloon acts as part of a safety system to capture the cryogenic cooling fluid in the event of a leak from the inner balloon. For the thermodynamics of the system to work well, it is beneficial that an outer surface of the inner balloon be in intimate contact with an inner surface of the outer balloon.

Attempts to control the intimate contact between the inner balloon and the outer balloon have not been altogether satisfactory. For example, one method utilizes a vacuum pump to evacuate a space between the inner balloon and the outer balloon through an exhaust pathway of the catheter. The evacuated space is then isolated from the exhaust pathway utilizing a check valve. The check valve is necessary due to varying pressure in the exhaust pathway from the space between the two balloons. However, due to a low, pressure differential across the check valve, the ability of the check valve to reliably maintain a separation between the two pathways is limited and the pressure of the evacuated space pressure can often change due to leakage across the check valve. Additionally, the use of a check valve in such a situation requires a check valve with a very low “cracking pressure”. Such check valves can be inherently unreliable due to the very small forces created by the low differential pressure. These types of check valves can be prone to leaking especially when subjected to any type of vibration or even small mechanical shocks.

### SUMMARY

The present invention is directed toward a cryogenic balloon catheter system for treating a condition in a patient. In various embodiments, the cryogenic balloon catheter system includes a balloon catheter and an inter-balloon pressure control assembly. The balloon catheter includes a first balloon; and a second balloon that substantially encircles the first balloon to define an inter-balloon space between the first balloon and the second balloon, the inter-balloon space having an inter-balloon pressure. The inter-balloon pressure control assembly controls the inter-balloon pressure in the inter-balloon space between the first balloon and the second balloon. The inter-balloon pressure control assembly includes (i) a vacuum pump that is configured to selectively evacuate a fluid from the inter-balloon space to adjust the inter-balloon pressure; and (ii) a solenoid valve that is in fluid communication with the inter-balloon space, the solenoid valve selectively allowing the vacuum pump to evacuate the fluid from the inter-balloon space.

In some embodiments, the solenoid valve is selectively movable between an open position where the solenoid valve allows the vacuum pump to evacuate the fluid from the inter-balloon space, and a closed position where the solenoid valve inhibits the vacuum pump from evacuating the fluid from the inter-balloon space. In such embodiments, the cryogenic balloon catheter system can further include a control system that controls movement of the solenoid valve between the open position and the closed position. More specifically, the control system can control movement of the solenoid valve between the open position and the closed position based at least in part on the inter-balloon pressure.

Additionally, in certain embodiments, the solenoid valve is selectively moved to the open position depending upon the inter-balloon pressure to allow the vacuum pump to decrease the inter-balloon pressure. In one such embodiment, moving the solenoid valve to the open position is configured to occur when the inter-balloon pressure falls outside a predetermined range. Further, in such embodiment, moving the solenoid valve to the closed position can be configured to occur when the inter-balloon pressure is maintained within the predetermined range. Alternatively, in another such embodiment, moving the solenoid valve to the open position is configured to occur when the inter-balloon pressure is maintained within a predetermined range. Further, in such embodiment, moving the solenoid valve to the closed position can be configured to occur when the inter-balloon pressure falls outside the predetermined range.

In some embodiments, the inter-balloon pressure control assembly further includes an inter-balloon pressure sensor that senses the inter-balloon pressure within the inter-balloon space. Additionally, the inter-balloon pressure sensor can be in fluid communication with the inter-balloon space.

Further, in certain embodiments, the cryogenic balloon catheter system further includes a handle assembly that is handled by an operator to control the balloon catheter. In some such embodiments, the solenoid valve is positioned within the handle assembly. Additionally, the inter-balloon pressure sensor can also be positioned within the handle assembly.

Moreover, in some embodiments, the cryogenic balloon catheter system further includes a control console. In certain such embodiments, the handle assembly is coupled to the control console. Additionally, in some such embodiments, the vacuum pump is positioned within the control console. Further, the solenoid valve can also be positioned within the control console.

The present invention is further directed toward a cryogenic balloon catheter system, comprising (A) a balloon catheter including a first balloon; and a second balloon that substantially encircles the first balloon to define an inter-balloon space between the first balloon and the second balloon, the inter-balloon space having an inter-balloon pressure; and (B) an inter-balloon pressure control assembly that controls the inter-balloon pressure in the inter-balloon space between the first balloon and the second balloon, the inter-balloon pressure control assembly including (i) a vacuum pump that is configured to selectively evacuate a fluid from the inter-balloon space to adjust the inter-balloon pressure; and (ii) a solenoid valve that is in fluid communication with the inter-balloon space, the solenoid valve selectively moving between (i) an open position wherein the vacuum pump evacuates the fluid from the inter-balloon space, and (ii) a closed position wherein the vacuum pump is inhibited from evacuating the fluid from the inter-balloon space.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a simplified schematic side view illustration of a patient and one embodiment of a cryogenic balloon catheter system including an inter-balloon pressure control assembly having features of the present invention;

FIG. 2 is a simplified schematic view illustration of a portion of the patient and a portion of an embodiment of the cryogenic balloon catheter system including one embodiment of the inter-balloon pressure control assembly; and

FIG. 3 is a simplified schematic view illustration of a portion of the patient and a portion of another embodiment of the cryogenic balloon catheter system including another embodiment of the inter-balloon pressure control assembly.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Embodiments of the present invention are described herein in the context of an inter-balloon pressure control assembly for use within a cryogenic balloon catheter system. In particular, the inter-balloon pressure control assembly is configured to provide pressure data and/or information to other structures within the cryogenic balloon catheter system, which can be used to control various functions of the cryogenic balloon catheter system.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound and laser energy, as non-exclusive examples. The present invention is intended to be effective with any or all of these and other forms of energy.

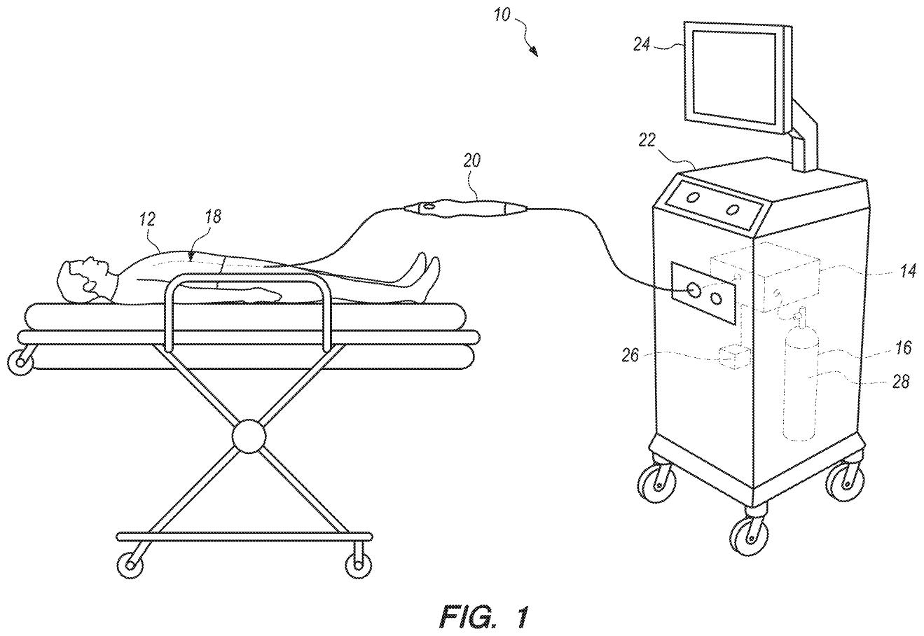

FIG. 1 is a simplified schematic side view illustration of an embodiment of a medical device 10 for use with a patient 12, which can be a human being or an animal. Although the specific medical device 10 illustrated and described herein pertains to and refers to a cryogenic balloon catheter system 10, it is understood and appreciated that other types of medical devices 10 or systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of a cryogenic balloon catheter system is not intended to be limiting in any manner.

The design of the cryogenic balloon catheter system 10 can be varied. In certain embodiments, such as the embodiment illustrated in FIG. 1, the cryogenic balloon catheter system 10 can include one or more of a control system 14 (illustrated in phantom), a fluid source 16 (illustrated in phantom), a balloon catheter 18, a handle assembly 20, a control console 22, a graphical display 24, and an inter-balloon pressure control assembly 26 (illustrated in phantom, and also sometimes referred to herein as a “pressure control assembly”).

It is understood that although FIG. 1 illustrates the structures of the cryogenic balloon catheter system 10 in a particular position, sequence and/or order, these structures can be located in any suitably different position, sequence and/or order than that illustrated in FIG. 1. It is also understood that the cryogenic balloon catheter system 10 can include fewer or additional components than those specifically illustrated and described herein.

In various embodiments, the control system 14 is configured to monitor and control various processes of the ablation procedure. More specifically, the control system 14 can monitor and control release and/or retrieval of a cooling fluid 28 (e.g., a cryogenic fluid) to and/or from the balloon catheter 18. The control system 14 can also control various structures that are responsible for maintaining and/or adjusting a flow rate and/or pressure of the cryogenic fluid 28 that is released to the balloon catheter 18 during the cryoablation procedure. In such embodiments, the cryogenic balloon catheter system 10 delivers ablative energy in the form of cryogenic fluid 28 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive data and/or other information (hereinafter sometimes referred to as “sensor output”) from various structures within the cryogenic balloon catheter system 10, and/or can receive data and/or other information (hereinafter sometimes referred to as “pressure control output”) from the pressure control assembly 26. In some embodiments, the control system 14 can receive, monitor, assimilate and/or integrate the sensor output, the pressure control output, and/or any other data or information received from any structure within the cryogenic balloon catheter system 10 in order to control the operation of the balloon catheter 18. As provided herein, in various embodiments, the control system 14 can initiate and/or terminate the flow of cryogenic fluid 28 to the balloon catheter 18 based on the sensor output and the pressure control output. Still further, or in the alternative, the control system 14 can control positioning of portions of the balloon catheter 18 within the body of the patient 12, and/or can control any other suitable functions of the balloon catheter 18.

The fluid source 16 contains the cryogenic fluid 28, which is delivered to the balloon catheter 18 with or without input from the control system 14 during a cryoablation procedure. Once the ablation procedure has initiated, the cryogenic fluid 28 can be delivered and the resulting gas, after a phase change, can be retrieved from the balloon catheter 18, and can either be vented or otherwise discarded as exhaust. Additionally, the type of cryogenic fluid 28 that is used during the cryoablation procedure can vary. In one non-exclusive embodiment, the cryogenic fluid 28 can include liquid nitrous oxide. However, any other suitable cryogenic fluid 28 can be used. For example, in one non-exclusive alternative embodiment, the cryogenic fluid 28 can include liquid nitrogen.

The design of the balloon catheter 18 can be varied to suit the specific design requirements of the cryogenic balloon catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a healthcare professional (also referred to herein as an “operator”). As used herein, a healthcare professional and/or an operator can include a physician, a physician's assistant, a nurse and/or any other suitable person and/or individual. In certain embodiments, the balloon catheter 18 is positioned within the body of the patient 12 utilizing at least a portion of the sensor output that is received by the control system 14. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue (not shown). While specific reference is made herein to the balloon catheter 18, as noted above, it is understood that any suitable type of medical device and/or catheter may be used.

The handle assembly 20 is handled and used by the operator to operate, position and control the balloon catheter 18. The design and specific features of the handle assembly 20 can vary to suit the design requirements of the cryogenic balloon catheter system 10. In the embodiment illustrated in FIG. 1, the handle assembly 20 is separate from, but in electrical and/or fluid communication with the control system 14, the fluid source 16, the graphical display 24, and the pressure control assembly 26. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In various embodiments, the handle assembly 20 can be used by the operator to initiate and/or terminate the cryoablation process, e.g., start the flow of the cryogenic fluid 28 to the balloon catheter 18 in order to ablate certain targeted heart tissue of the patient 12. In certain embodiments, the control system 14 can override use of the handle assembly 20 by the operator. Stated in another manner, in some embodiments, based at least in part on the pressure control output, the control system 14 can terminate the cryoablation process without the operator using the handle assembly 20 to do so.

The control console 22 is coupled to balloon catheter 18 and the handle assembly 20. Additionally, in the embodiment illustrated in FIG. 1, the control console 22 includes at least a portion of the control system 14, the fluid source 16, the graphical display 24, and the pressure control assembly 26. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the graphical display 24.

In various embodiments, the graphical display 24 is electrically connected to the control system 14 and the pressure control assembly 26. Additionally, the graphical display 24 provides the operator of the cryogenic balloon catheter system 10 with information that can be used before, during and after the cryoablation procedure. For example, the graphical display 24 can provide the operator with information based on the sensor output, the pressure control output, and any other relevant information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

The inter-balloon pressure control assembly 26 can be positioned in any suitable manner within the cryogenic balloon catheter system 10. For example, as illustrated in FIG. 1, in certain embodiments, at least a portion of the inter-balloon pressure control assembly 26 can be positioned within the control console 22 and/or adjacent to the control system 14. Additionally, or in the alternative, in some embodiments, at least a portion of the inter-balloon pressure control assembly 26 can be positioned within and/or substantially adjacent to the handle assembly 20. Further, or in the alternative, the inner-balloon pressure control assembly 26 can be positioned in another suitable manner at any suitable location(s) within the cryogenic balloon catheter system 10.

As provided herein, the inter-balloon pressure control assembly 26 can sense, monitor and/or control an inter-balloon pressure within a portion of the balloon catheter 18. Further, the inter-balloon pressure control assembly 26 can provide pressure data and/or information to other structures within the cryogenic balloon catheter system 10, e.g., the control system 14, which can be used to control various functions of the cryogenic balloon catheter system 10 as described herein. The various components and modes of operation of embodiments of the pressure control assembly 26 will be described in greater detail herein below.

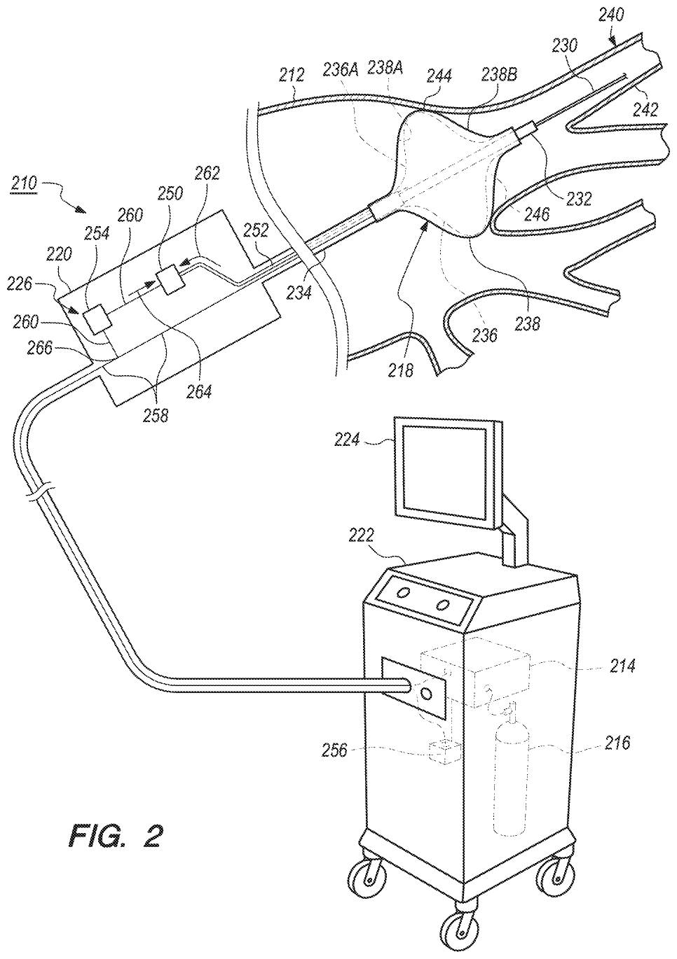

FIG. 2 is a simplified schematic view illustration of a portion of one embodiment of the cryogenic balloon catheter system 210 and a portion of a patient 212. In the embodiment illustrated in FIG. 2, the cryogenic balloon catheter system 210 includes one or more of a control system 214 (illustrated in phantom), a fluid source 216 (illustrated in phantom), a balloon catheter 218, a handle assembly 220, a control console 222, a graphical display 224, and an inter-balloon pressure control assembly 226 (also sometimes referred to herein as a “pressure control assembly”).

The control system 214 is configured to control various functions of the cryogenic balloon catheter system 210. As shown in FIG. 2, in certain embodiments, the control system 214 can be positioned substantially within the control console 222. Alternatively, at least a portion of the control system 214 can be positioned in one or more other locations within the cryogenic balloon catheter system 210, e.g., within the handle assembly 220. In one embodiment, the control system 214 can control various functions of the remainder of the cryogenic balloon catheter system 210 based at least in part on data or other information received by the control system 214, as provided in greater detail herein.

The design of the balloon catheter 218 can be varied to suit the design requirements of the cryogenic balloon catheter system 210. In this embodiment, the balloon catheter 218 includes one or more of a guidewire 230, a guidewire lumen 232, a catheter shaft 234, an inner balloon 236 (sometimes referred to herein simply as a “first balloon”) and an outer balloon 238 (sometimes referred to herein simply as a “second balloon”). It is understood that the balloon catheter 218 can include other structures as well. However, for the sake of clarity, these other structures have been omitted from the Figures. In the embodiment illustrated in FIG. 2, the balloon catheter 218 is positioned within the circulatory system 240 of the patient 212. The guidewire 230 and guidewire lumen 232 are inserted into a pulmonary vein 242 of the patient 212, and the catheter shaft 234 and the balloons 236, 238 are moved along the guidewire 230 and/or the guidewire lumen 232 to near an ostium 244 of the pulmonary vein 242.

In one embodiment, the inner balloon 236 can be made from a relatively non-compliant or semi-compliant material. Some representative materials suitable for this application include PET (polyethylene terephthalate), nylon, polyurethane, and copolymers of these materials such as polyether block amide (PEBA), known under its trade name as PEBAX® (supplier Arkema), as non-exclusive examples. In another embodiment, a polyester block copolymer known in the trade as Hytrel® (DuPont™) is also a suitable material for the inner balloon 236. The inner balloon 236 can be relatively inelastic in comparison to the outer balloon 238.

The outer balloon 238 substantially encircles the inner balloon 236. In certain embodiments, the outer balloon 238 can be made from a relatively compliant material. Such materials are well known in the art. One non-exclusive example is aliphatic polyether polyurethanes in which carbon atoms are linked in open chains, including paraffins, olefins, and acetylenes. Another available example goes by the trade name Tecoflex® (Lubrizol). Other available polymers from the polyurethane class of thermoplastic polymers with exceptional elongation characteristics are also suitable for use as the outer balloon 238. In one embodiment, either of the balloons 236, 238, may be rendered electrically conductive by doping the material from which it is made with a conductive metal or other conductive substance. In such embodiment, the electrically conductive balloons can be particularly suitable for the outer balloon 238.

During use, the inner balloon 236 can be partially or fully inflated so that at least a portion of the inner balloon 236 expands against at least a portion of the outer balloon 238. Stated in another manner, during use of the balloon catheter 218, at least a portion of an outer surface 236A of the inner balloon 236 expands and is positioned substantially directly against a portion of an inner surface 238A of the outer balloon 238. At certain times during usage of the cryogenic balloon catheter system 210, the inner balloon 236 and the outer balloon 238 define an inter-balloon space 246, or gap, between the balloons 236, 238. The inter-balloon space 246 is illustrated between the inner balloon 236 and the outer balloon 238 in FIG. 2 for clarity, although it is understood that at certain times during usage of the cryogenic balloon catheter system 210, the inter-balloon space 246 has very little or no volume. As provided herein, once the inner balloon 236 is sufficiently inflated, an outer surface 238B of the outer balloon 238 can then be positioned within the circulatory system 240 of the patient 212 to abut and/or substantially form a seal with the ostium 244 of the pulmonary vein 242 to be treated.

The design of the handle assembly 220 can vary. In certain embodiments, the handle assembly 220 can include circuitry (not shown in FIG. 2) that can include at least a portion of the control system 214. Alternatively, the circuitry can transmit electrical signals such as the sensor output and/or the pressure control output, or otherwise provide data to the control system 214 as described herein. Additionally, or in the alternative, the circuitry can receive electrical signals or data from the inter-balloon pressure control assembly 226. In one embodiment, the circuitry can include a printed circuit board having one or more integrated circuits, or any other suitable circuitry.

The inter-balloon pressure control assembly 226 senses, adjusts, controls and/or monitors an inter-balloon pressure between the inner balloon 236 and the outer balloon 238. As used herein, the “inter-balloon pressure” means the pressure inside of the inter-balloon space 246 at or substantially contemporaneously with the time the pressure in the inter-balloon space 246 is measured. In the embodiment illustrated in FIG. 2, the inter-balloon pressure control assembly 226 can transmit electrical signals and/or other forms of data or information to the control system 214.

The design of the inter-balloon pressure control assembly 226 can be varied. In the embodiment illustrated in FIG. 2, the inter-balloon pressure control assembly 226 includes an inter-balloon pressure sensor 250, an inter-balloon tubular member 252, a solenoid valve 254, a vacuum pump 256, a vacuum exhaust line 258 and an inter-balloon space exhaust line 260.

The inter-balloon pressure sensor 250 senses and/or monitors the inter-balloon pressure within the inter-balloon space 246. The type of inter-balloon pressure sensor 250 that is used can vary depending upon the design requirements of the cryogenic balloon catheter system 210 and/or the inter-balloon pressure control assembly 226. For example, in one embodiment, the inter-balloon pressure sensor 250 can include a “MEMS” sensor or an optical pressure detector, as nonexclusive examples. Alternatively, another suitable type of inter-balloon pressure sensor 250 can be used.

In the embodiment illustrated in FIG. 2, the inter-balloon pressure sensor 250 is positioned within the handle assembly 220. In an alternative embodiment, the inter-balloon pressure sensor 250 can be positioned anywhere between the inter-balloon space 246 and the handle assembly 220. Still alternatively, the inter-balloon pressure sensor 250 can be positioned between the handle assembly 220 and the control console 222. In another embodiment, the inter-balloon pressure sensor 250 can be positioned within the control console 222. As set forth in greater detail here, in certain embodiments, the inter-balloon pressure sensor 250 can incorporate the use of the inter-balloon tubular member 252.

In the embodiment illustrated in FIG. 2, the inter-balloon tubular member 252 extends from the inter-balloon pressure sensor 250 to the inter-balloon space 246. The inter-balloon pressure sensor 250 is in fluid communication with the inter-balloon space 246 via the inter-balloon tubular member 252. The inter-balloon tubular member 252 can be a relatively small diameter tube that can transmit the inter-balloon pressure within the inter-balloon space 246 directly to the inter-balloon pressure sensor 250. As the inter-balloon pressure sensor 250 determines, senses and/or monitors the inter-balloon pressure, the inter-balloon pressure sensor 250 can then send a sensor output and/or a pressure control output, e.g., electrical signals regarding the inter-balloon pressure, to the control console 222, i.e. the control system 214.

The solenoid valve 254 is in fluid communication with the inter-balloon space 246. Additionally, the solenoid valve 254 selectively allows the vacuum pump 256 to evacuate the inter-balloon space 246 of any fluid which may be present between the inner balloon 236 and the outer balloon 238. Further, as provided herein, the solenoid valve 254 is selectively movable, e.g., under control of the control system 214, between an open position and a closed position.

When the solenoid valve 254 is in the open position, the solenoid valve 254 allows the vacuum pump 256 to evacuate fluid from the inter-balloon space 246, and thus to decrease the inter-balloon pressure. In some embodiments, the solenoid valve 254 is configured to be moved to the open position when the inter-balloon pressure, e.g., as sensed by the inter-balloon pressure sensor 250, falls outside a predetermined range. Alternatively, in other embodiments, the solenoid valve 254 is configured to be moved to the open position when the inter-balloon pressure, e.g., as sensed by the inter-balloon pressure sensor 250, is maintained within a predetermined range. Additionally, in one embodiment, once the inter-balloon space 246 has been evacuated, the solenoid valve 254 can be moved to the closed position.

Conversely, when the solenoid valve 254 moves to the closed position, the solenoid valve 254 inhibits the vacuum pump 256 from evacuating fluid from the inter-balloon space 246, or the inter-balloon space 246 has already been evacuated so there may be no need to continue to pull a vacuum on the inter-balloon space 246 at that time. In some embodiments, the solenoid valve 254 is configured to be moved to the closed position when the inter-balloon pressure, e.g., as sensed by the inter-balloon pressure sensor 250, is maintained within a predetermined range. Alternatively, in other embodiments, the solenoid valve 254 is configured to be move to the closed position when the inter-balloon pressure, e.g., as sensed by the inter-balloon pressure sensor 250, falls outside a predetermined range.

The solenoid valve 254 can be controlled by the control system 214, i.e. between the open position and the closed position, based at least in part on the sensor output and/or the pressure control output (e.g., the inter-balloon pressure) received from the inter-balloon pressure sensor 250. In the embodiment illustrated in FIG. 2, the solenoid valve 254 is positioned within the handle assembly 220. However, in non-exclusive alternative embodiments, the solenoid valve 254 can be positioned in other suitable locations outside of the handle assembly 220.

As provided herein, the vacuum pump 256 is configured to selectively evacuate fluid from the inter-balloon space 246, i.e. under control of the control system 214. In certain embodiments, as shown in FIG. 2, the vacuum pump 256 can be positioned within the control console 222. Alternatively, the vacuum pump 256 can be positioned in another suitable location within the cryogenic balloon catheter system 210. In the embodiment illustrated in FIG. 2, the inter-balloon space exhaust line 260 extends from the inter-balloon pressure sensor 250 to the solenoid valve 254, and from the solenoid valve 254 to the vacuum exhaust line 258. Therefore, in this embodiment, the inter-balloon space exhaust line 260 is used in conjunction with the inter-balloon tubular member 252 to provide an avenue for any fluid to move from the inter-balloon space 246 to the vacuum exhaust line 258 in the direction of arrow 262 in order to decrease the inter-balloon pressure. In an alternative embodiment, the inter-balloon space exhaust line 260 and the inter-balloon tubular member 252 can provide an avenue for fluid to move to the inter-balloon space 246 in the direction of arrow 264 in order to increase the inter-balloon pressure should that be required or desired during a cryoablation procedure. In the embodiment illustrated in FIG. 2, the solenoid valve 254 can be tied in to the vacuum exhaust line 258 at an exhaust line junction 266 via the inter-balloon space exhaust line 260. In this embodiment, the vacuum pump 256 can also generate a vacuum to remove cooling fluid 28 (illustrated in FIG. 1) from an interior of the inner balloon 236 via the vacuum exhaust line 258.

In certain embodiments, the control system 214 is configured to process and integrate the sensor output and/or the pressure control output, e.g., from the inter-balloon pressure sensor 250, to determine and/or adjust for proper functioning of the cryogenic balloon catheter system 210. Based at least in part on the sensor output and/or the pressure control output, the control system 214 can determine that certain modifications to the functioning of the cryogenic balloon catheter system 210 are required, such as opening or closing of the solenoid valve 254. When the solenoid valve 254 is open, the inter-balloon pressure decreases until the desired inter-balloon pressure is reached. When the solenoid valve 254 is closed, a sealed volume of the inter-balloon space 246 occurs. By actively opening and/or closing the solenoid valve 254, a desired inter-balloon pressure and/or volume of the inter-balloon space 246 can be maintained.

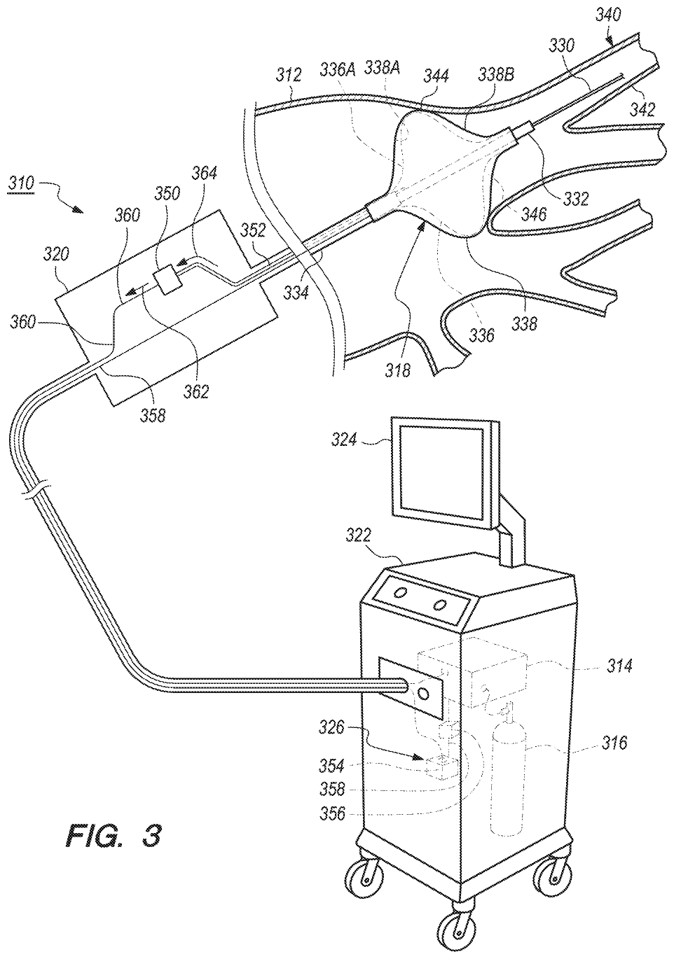

FIG. 3 is a simplified schematic view illustration of a portion of another embodiment of the cryogenic balloon catheter system 310 and a portion of a patient 312. As shown in FIG. 3, the cryogenic balloon catheter system 310 is somewhat similar to the embodiment of the cryogenic balloon catheter system 210 illustrated and described in relation to FIG. 2. For example, in the embodiment illustrated in FIG. 3, the cryogenic balloon catheter system 310 again includes a control system 314 (illustrated in phantom), a fluid source 316 (illustrated in phantom), a balloon catheter 318, a handle assembly 320, a control console 322, a graphical display 324, and an inter-balloon pressure control assembly 326 (illustrated in phantom).

As with the previous embodiment, the control system 314 is configured to control various functions of the cryogenic balloon catheter system 310. As shown in FIG. 3, in certain embodiments, the control system 314 can be positioned substantially within the control console 322. Alternatively, at least a portion of the control system 314 can be positioned in one or more other locations within the cryogenic balloon catheter system 210, e.g., within the handle assembly 320. In one embodiment, the control system 314 can control various functions of the remainder of the cryogenic balloon catheter system 310 based at least in part on data or other information received by the control system 314, as provided in greater detail herein.

The design of the balloon catheter 318 can be varied to suit the design requirements of the cryogenic balloon catheter system 310. In this embodiment, the balloon catheter 318 includes one or more of a guidewire 330, a guidewire lumen 332, a catheter shaft 334, an inner balloon 336 (sometimes referred to herein simply as a “first balloon”) and an outer balloon 338 (sometimes referred to herein simply as a “second balloon”). It is understood that the balloon catheter 318 can include other structures as well. However, for the sake of clarity, these other structures have been omitted from the Figures. In the embodiment illustrated in FIG. 3, the balloon catheter 318 is again positioned within the circulatory system 340 of the patient 312. The guidewire 330 and guidewire lumen 332 are inserted into a pulmonary vein 342 of the patient 312, and the catheter shaft 334 and the balloons 336, 338 are moved along the guidewire 330 and/or the guidewire lumen 332 to near an ostium 344 of the pulmonary vein 342.

During use, the inner balloon 336 can be partially or fully inflated so that at least a portion of the inner balloon 336 expands against at least a portion of the outer balloon 338. Stated in another manner, during use of the balloon catheter 318, at least a portion of an outer surface 336A of the inner balloon 336 expands and is positioned substantially directly against a portion of an inner surface 338A of the outer balloon 338. At certain times during usage of the cryogenic balloon catheter system 310, the inner balloon 336 and the outer balloon 338 define an inter-balloon space 346, or gap, between the balloons 336, 338. The inter-balloon space 346 is illustrated between the inner balloon 336 and the outer balloon 338 in FIG. 3 for clarity, although it is understood that at certain times during usage of the cryogenic balloon catheter system 310, the inter-balloon space 346 has very little or no volume. As provided herein, once the inner balloon 336 is sufficiently inflated, an outer surface 338B of the outer balloon 338 can then be positioned within the circulatory system 340 of the patient 312 to abut and/or substantially form a seal with the ostium 344 of the pulmonary vein 342 to be treated.

The design of the handle assembly 320 can vary. In certain embodiments, the handle assembly 320 can include circuitry (not shown in FIG. 3) that can include at least a portion of the control system 314. Alternatively, the circuitry can transmit electrical signals such as the sensor output and/or the pressure control output, or otherwise provide data to the control system 314 as described herein. Additionally, or in the alternative, the circuitry can receive electrical signals or data from the inter-balloon pressure control assembly 326. In one embodiment, the circuitry can include a printed circuit board having one or more integrated circuits, or any other suitable circuitry.

The inter-balloon pressure control assembly 326 senses, adjusts, controls and/or monitors an inter-balloon pressure between the inner balloon 336 and the outer balloon 338. In the embodiment illustrated in FIG. 3, the inter-balloon pressure control assembly 326 can transmit electrical signals and/or other forms of data or information to the control system 314. The design of the inter-balloon pressure control assembly 326 can be varied. In the embodiment illustrated in FIG. 3, the inter-balloon pressure control assembly 326 includes an inter-balloon pressure sensor 350, an inter-balloon tubular member 352, a solenoid valve 354, a vacuum pump 356, a vacuum exhaust line 358 and an inter-balloon space exhaust line 360.

The inter-balloon pressure sensor 350 senses and/or monitors the inter-balloon pressure within the inter-balloon space 346. The type of inter-balloon pressure sensor 350 that is used can vary depending upon the design requirements of the cryogenic balloon catheter system 310 and/or the inter-balloon pressure control assembly 326.

In the embodiment illustrated in FIG. 3, the inter-balloon pressure sensor 350 is positioned within the handle assembly 320. In an alternative embodiment, the inter-balloon pressure sensor 350 can be positioned anywhere between the inter-balloon space 346 and the handle assembly 320. Still alternatively, the inter-balloon pressure sensor 350 can be positioned between the handle assembly 320 and the control console 322. In another embodiment, the inter-balloon pressure sensor 350 can be positioned within the control console 322. As set forth in greater detail here, in certain embodiments, the inter-balloon pressure sensor 350 can incorporate the use of the inter-balloon tubular member 352.

In the embodiment illustrated in FIG. 3, the inter-balloon tubular member 352 extends from the inter-balloon pressure sensor 350 to the inter-balloon space 346. The inter-balloon pressure sensor 350 is in fluid communication with the inter-balloon space 346 via the inter-balloon tubular member 352. The inter-balloon tubular member 352 can be a relatively small diameter tube that can transmit the inter-balloon pressure within the inter-balloon space 346 directly to the inter-balloon pressure sensor 350. As the inter-balloon pressure sensor 350 determines, senses and/or monitors the inter-balloon pressure, the inter-balloon pressure sensor 350 can then send a sensor output and/or a pressure control output, e.g., electrical signals regarding the inter-balloon pressure, to the control console 322, i.e. the control system 314.

The solenoid valve 354 is in fluid communication with the inter-balloon space 346. Additionally, the solenoid valve 354 selectively allows the vacuum pump 356 to evacuate the inter-balloon space 346 of any fluid which may be present between the inner balloon 336 and the outer balloon 338. Further, as provided herein, the solenoid valve 354 is selectively movable, e.g., under control of the control system 314, between an open position and a closed position.

When the solenoid valve 354 is in the open position, the solenoid valve 354 allows the vacuum pump 356 to evacuate fluid from the inter-balloon space 346. Conversely, when the solenoid valve 354 moves to the closed position, the solenoid valve 354 inhibits the vacuum pump 356 from evacuating fluid from the inter-balloon space 346. The solenoid valve 354 can be controlled by the control system 314, i.e. between the open position and the closed position, based at least in part on the sensor output and/or the pressure control output (e.g., the inter-balloon pressure) received from the inter-balloon pressure sensor 350. In the embodiment illustrated in FIG. 3, the solenoid valve 354 is positioned within the control console 322. However, in non-exclusive alternative embodiments, the solenoid valve 354 can be positioned in other locations outside of the control console 322.

As provided herein, the vacuum pump 356 is configured to selectively evacuate fluid from the inter-balloon space 346, i.e. under control of the control system 314. In certain embodiments, as shown in FIG. 3, the vacuum pump 356 can be positioned within the control console 322. Alternatively, the vacuum pump 356 can be positioned in another suitable location within the cryogenic balloon catheter system 310.

In the embodiment illustrated in FIG. 3, the inter-balloon space exhaust line 360 extends from the inter-balloon pressure sensor 350 to the solenoid valve 354, and from the solenoid valve 354 to the vacuum exhaust line 358. Therefore, in this embodiment, the inter-balloon space exhaust line 360 is used in conjunction with the inter-balloon tubular member 352 to provide an avenue for any fluid to move from the inter-balloon space 346 to an exhaust (not shown) of the vacuum pump 356 in the direction of arrow 362 in order to decrease the inter-balloon pressure. In an alternative embodiment, the inter-balloon space exhaust line 360 and the inter-balloon tubular member 352 can provide an avenue for fluid to move to the inter-balloon space 346 in the direction of arrow 364 in order to increase the inter-balloon pressure should that be required or desired during a cryoablation procedure. In one embodiment, the vacuum pump 356 can also generate a vacuum to remove cooling fluid 28 (illustrated in FIG. 1) from an interior of the inner balloon 336 via the vacuum exhaust line 358.

In certain embodiments, the control system 314 is configured to process and integrate the sensor output and/or the pressure control output, e.g., from the inter-balloon pressure sensor 350, to determine and/or adjust for proper functioning of the cryogenic balloon catheter system 310. Based at least in part on the sensor output and/or the pressure control output, the control system 314 can determine that certain modifications to the functioning of the cryogenic balloon catheter system 310 are required, such as opening or closing of the solenoid valve 354. When the solenoid valve 354 is open, the inter-balloon pressure decreases until the desired inter-balloon pressure is reached. When the solenoid valve 354 is closed, a sealed volume of the inter-balloon space 346 occurs. By actively opening and/or closing the solenoid valve 354, a desired inter-balloon pressure and/or volume of the inter-balloon space 346 can be maintained. In another embodiment, the control system 314 can cause the solenoid valve 354 to open and/or close based on time, rather than on the sensor output. In still another embodiment, the sensor output, the pressure control output and time can be used by the control system 314 in order to open and/or close the solenoid valve 354.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A cryogenic balloon catheter system, comprising:
a balloon catheter including a shaft and a cryoballoon attached to the shaft the cryoballoon including a first balloon, and a second balloon that substantially encircles the first balloon to define an inter-balloon space between the first balloon and the second balloon, the inter-balloon space having an inter-balloon pressure; and
an inter-balloon pressure control assembly that controls the inter-balloon pressure, the inter-balloon pressure control assembly including:
an inter-balloon pressure sensor configured to sense the inter-balloon pressure and generate a sensor output responsive thereto;
an inter-balloon tubular member in fluid communication with the inter-balloon space and the inter-balloon pressure sensor;
a vacuum pump; and
a solenoid in fluid communication with the inter-balloon space and the vacuum pump, wherein the solenoid valve is configured to selectively permit fluid communication between the vacuum pump and the inter-balloon space such that the vacuum pump can evacuate a fluid from the inter-balloon space to adjust the inter-balloon pressure based on the sensor output.

2. The cryogenic balloon catheter system of claim 1, wherein the solenoid valve is selectively controllable between an open state that allows the vacuum pump to evacuate the fluid from the inter-balloon space, and a closed state that inhibits the vacuum pump from evacuating the fluid from the inter-balloon space.

3. The cryogenic balloon catheter system of claim 2, wherein the solenoid valve is configured to assume the open state when the inter-balloon pressure falls outside a predetermined range.

4. The cryogenic balloon catheter system of claim 3, wherein the solenoid valve is configured to assume the closed state when the inter-balloon pressure is maintained within the predetermined range.

5. The cryogenic balloon catheter system of claim 2, wherein the solenoid valve is configured to assume the open state when the inter-balloon pressure is within a predetermined range.

6. The cryogenic balloon catheter system of claim 5, wherein the solenoid valve is configured to assume the closed state when the inter-balloon pressure falls outside the predetermined range.

7. The cryogenic balloon catheter system of claim 1, further comprising a handle assembly attached to the shaft and operable by a user to control the balloon catheter.

8. The cryogenic balloon catheter system of claim 7, wherein the inter-balloon pressure sensor is positioned within the handle assembly, and wherein the inter-balloon tubular member extends from the inter-balloon space through the shaft and into the handle assembly.

9. The cryogenic balloon catheter system of claim 8, wherein the solenoid valve is positioned within the handle assembly.

10. A cryogenic balloon catheter system, comprising:
a balloon catheter including a shaft, a handle assembly attached to the shaft, and a cryoballoon attached to the shaft, the cryoballoon including a first balloon, and a second balloon that substantially encircles the first balloon to define an inter-balloon space between the first balloon and the second balloon, the inter-balloon space having an inter-balloon pressure; and
an inter-balloon pressure sensor configured to sense the inter-balloon pressure and generate a sensor output responsive thereto;
an inter-balloon tubular member in fluid communication with the inter-balloon space and the inter-balloon pressure sensor;
a control system operatively coupled to the inter-balloon pressure sensor;
a vacuum pump; and
a solenoid operatively coupled to the control system and in fluid communication with the inter-balloon space and the vacuum pump,
wherein the control system is configured to control the solenoid valve based on the sensor output to selectively permit fluid communication between the vacuum pump and the inter-balloon space such that the vacuum pump can evacuate a fluid from the inter-balloon space.

11. The cryogenic balloon catheter system of claim 10, wherein the control system is configured to open the solenoid valve responsive to the sensor output indicating that the inter-balloon pressure falls outside a predetermined range.

12. The cryogenic balloon catheter system of claim 10, wherein the control system is configured to maintain the solenoid valve in an open state when the sensor output indicates that the inter-balloon pressure is within a predetermined range.

13. The cryogenic balloon catheter system of claim 10, wherein the inter-balloon pressure sensor is positioned within the handle assembly, and wherein the inter-balloon tubular member extends from the inter-balloon space through the shaft and into the handle assembly so as to fluidly couple the inter-balloon space and the inter-balloon pressure sensor.

14. The cryogenic balloon catheter system of claim 13, wherein the solenoid valve is positioned within the handle assembly.

15. The cryogenic balloon catheter system of claim 13, further comprising a control console, wherein the solenoid valve and the vacuum pump are positioned within the control console.

16. The cryogenic balloon catheter system of claim 15, further comprising a vacuum exhaust line fluidly coupled to the vacuum pump for removal of fluid from an interior of the first balloon, and an inter-balloon exhaust line fluidly coupled to the vacuum exhaust line.

17. A method of controlling pressure within a cryogenic balloon catheter, the cryogenic balloon catheter including a handle assembly, a shaft attached to the handle assembly, and a cryoballoon attached to the shaft, the cryoballoon including a first balloon and a second balloon that substantially encircles the first balloon to define an inter-balloon space between the first balloon and the second balloon, the method comprising:
sensing an inter-balloon pressure within the inter-balloon space using an inter-balloon pressure sensor positioned within the handle assembly, the inter-balloon pressure sensor generating a sensor output based on the sensed inter-balloon pressure;
responsive to the sensor output, controlling a solenoid valve between a closed state in which the solenoid valve fluidly isolates the inter-balloon space from a vacuum pump, and an open state in which the inter-balloon space is fluidly coupled to the vacuum pump through the solenoid valve.

18. The method of claim 17, wherein controlling the solenoid valve includes causing the solenoid valve to assume the open state when the inter-balloon pressure falls outside a predetermined range.

19. The method of claim 18, wherein controlling the solenoid valve further includes maintaining the solenoid valve in the closed state when the inter-balloon pressure is within the predetermined range.

20. The method of claim 17, further comprising removing fluid from an interior of the first balloon through a vacuum exhaust line fluidly coupled to the interior of the first balloon and the vacuum pump, and evacuating fluid from the inter-balloon space through an inter-balloon space exhaust line fluidly coupled to the vacuum exhaust line through the solenoid valve.
